(19) United States
(12) Patent Application Publication
(10) Pub. No.: US 2026/0256350 A1
(43) Pub. Date: Sep. 3, 2026

(54) EXTRA-ORAL IMAGE TAKING KIT

(71) Applicant: DENTAL MONITORING, PARIS (FR)

(72) Inventors: Thomas PELLISSARD, PARIS (FR); Cédric LANÇON, VILLERS FARLAY (FR); Louis-Charles ROISIN, PARIS (FR)

(21) Appl. No.: 18/880,092

(22) PCT Filed: Jun. 30, 2023
(86) PCT No.: PCT/EP2023/068115

§ 371 Date: Dec. 30, 2024

(30) Foreign Application Priority Data
Jul. 4, 2022 (FR) FR2206745

(51) Int. Cl. A61B 1/24; A61B 1/00

(52) U.S. Cl. CPC A61B 1/24; A61B 1/00006; A61B 1/00016; A61B 1/00045

(57) ABSTRACT

An image taking kit comprising: an extra-oral image taking device comprising a support provided with a mouthpiece defining a mouthpiece opening, an image acquisition apparatus attached to the support and comprising a camera for acquiring at least one image of a dental scene seen through the mouthpiece opening, and a display device comprising a screen for displaying an image representing the scene acquired by the image acquisition apparatus, the image acquisition apparatus comprising an additional screen and/or the support defining an image taking chamber, the image acquisition apparatus being at least partially, preferably entirely, accommodated in the image taking chamber.

The present invention relates to a kit for taking images of dental scenes from a vantage point outside the mouth and for visualizing the corresponding images.

It is known, for example from WO 2016/066651A1, to monitor the progress of a patient's orthodontic treatment by comparing the evolution of an orthodontic model generated from images of the patient's lower and upper dental arches acquired prior to the implementation of the orthodontic treatment with images of said arches acquired during the course of the orthodontic treatment.

Images of the dental arches can be acquired using an image taking device comprising an image acquisition apparatus and a mouthpiece. Examples of such image taking devices are described in EP 3 391 810 A1 and WO 2022/008533A1, which comprise a tubular, hollow support, carrying a mouthpiece at one end, and at the opposite end of which is mounted a cell phone equipped with a camera directed towards the patient's mouth to take images of the dental arches. The images can then be transmitted to an orthodontist or dentist, who can analyze them either directly or after appropriate digital processing.

However, the patient using the devices of EP 3 391 810 A1 and WO 2022/008533 A1 cannot directly observe the images they are acquiring on the cell phone screen, as the cell phone camera is positioned on the side opposite the screen. In addition, to order an image to be taken, they may need to press the phone's touch screen. To get around these problems, they must use a mirror, which is impractical.

There is therefore a need to remedy this shortcoming.

The invention proposes a image taking kit comprising:
a device for extra-oral photography comprising:
a support,
a mouthpiece that defines a mouthpiece opening and is carried by the support,
an image acquisition apparatus attached to the support and comprising a camera for acquiring at least one image of a dental scene seen through the mouthpiece opening, and
a display device comprising a screen for displaying an image representing the scene acquired by the image acquisition apparatus,
the image acquisition apparatus preferably comprising an additional screen and/or the support defining an image taking chamber, the image acquisition apparatus preferably being at least partially, preferably entirely, accommodated in the image taking chamber.

The user of the kit can thus directly view the image acquired by the image acquisition apparatus immediately after its acquisition. For example, with the mouthpiece positioned in their mouth, the user can easily check the quality, e.g. contrast and/or brightness, of the image, and/or can move the image taking device relative to their dental arches, so as to acquire an image from another vantage point, e.g. one better suited to representing the dental scene.

In addition, use of the kit according to the invention can reduce the light pollution induced by the use of a mirror. Mirror observation, as described above, is generally carried out in a bathroom, where specific, powerful lighting is usually associated with the mirror and can interfere with image acquisition.

An “extra-oral” image is acquired using the image taking device, which in an operating configuration in which the mouthpiece is inserted into the user's mouth, is such that the image acquisition apparatus is outside the user's oral cavity. An extra-oral image is acquired from a position distant from the oral cavity.

The display device is preferably movable relative to the image taking device.

The display device can be chosen from a cell phone, a digital tablet, a desktop computer, in particular a transportable one, or a television, in particular a connected one. The display screen can be touch-sensitive.

Preferably, the kit comprises a communication module for transferring images acquired by the image taking device to the display device. The communication module can be a wireless communication module, in particular over the air, for example using the Wifi® or Bluetooth® data transfer protocol. In particular, the image taking device may comprise a wave transmitter for sending an image acquired by the image acquisition apparatus, and the display device may comprise a wave receiver for receiving said image.

In one embodiment, the communication module comprises a data transmission cable, for example of the USB type, for connecting the image taking device to the display device, and the image taking device and the display device each comprise a connection socket for plugging in the data transmission cable.

The kit may further comprise a control module to trigger image acquisition. The image-taking device and/or the display device may comprise the control module.

The control module preferably comprises a computer program comprising instructions for guiding the user to position the image taking device in at least one image acquisition position, and a processor for executing the computer program. In particular, in the variant in which the acquisition device is accommodated in the image taking chamber, the control module is accommodated in the display device.

The computer program may comprise instructions for processing the acquired image(s), in order to provide at least one piece of information to the user. The information can be audible, for example in the form of a voice message, in order to position the device relative to the user's dental arches to attain a predetermined acquisition position and/or orientation, and optionally, specify to the user the number of images to be acquired. If required, the image taking device and/or display device may further comprise a speaker for broadcasting information. The information can be visual and can be displayed on the screen, alongside or visually superimposed atop the displayed image.

The information can be selected from the number of images to be acquired, information relating to the setting of the support geometry, the orientation of the support and/or the positioning of the user's mouth on the mouthpiece.

The computer program may further comprise instructions for requesting and/or waiting for a user action, for example to trigger image acquisition. For example, after execution by the processor, the computer program may require pressing on an area of the touchscreen of the display device. In another example, it may wait for a voice command from the user. In particular for this purpose, the image taking device and/or display device may comprise a microphone.

The control module may comprise an actuator and be configured to trigger image acquisition by the acquisition device following actuation of the actuator. For example, the actuator is electrically connected to the acquisition device. The actuator is, for example, a pushbutton. It can be carried by the support.

The control module can be configured to detect the presence of a pattern and/or object in the scene viewed by the camera, and to trigger image acquisition as soon as the pattern and/or object is detected. Pattern and/or object detection is performed, for example, using a deep learning algorithm or a computer vision technique.

The control module is preferably configured to trigger the transfer of the image(s) acquired by the image taking device to the display device. It can be configured so that image transfer is carried out in real time, i.e. so that the screen displays the image acquired by the acquisition device in real time. “Real time” is understood to mean that the time between acquisition and display of an image is less than 1 s, or even less than 500 ms, or even less than 10 ms.

According to one variant where the display device includes the control module, the control module can notably send at least one control signal to the image taking device via the communication module.

The image taking device and/or display device may comprise a storage module, such as a hard disk or Flash memory, to digitally store the images acquired by the acquisition apparatus.

The camera may have a focal length of between 25 mm and 90 mm, preferably between 30 mm and 45 mm.

The camera's focal length may be fixed, making it possible to use a compact camera. In one variant, the camera may comprise an autofocus system that varies the focal length.

The camera may extend along an axis parallel to, or even coincident with, the longitudinal axis of the support. The line of sight of the camera may be parallel to, or even coincident with, the longitudinal axis of the support.

The image acquisition apparatus may comprise, or even consist of, a plurality of cameras. The cameras can be arranged along different lines of sight, and to each acquire different views of the same dental scene. The image acquisition apparatus can thus provide a stereoscopic view of the dental scene. The cameras may have the same focal length.

The image acquisition apparatus may be fixed relative to the mouthpiece. In this way, the image taking device enables images to be taken under similar conditions at different times during the orthodontic process. Similar acquisition conditions comprise the position and relative orientation of the image acquisition apparatus with respect to the patient's teeth. In one embodiment, the image acquisition apparatus can be movable relative to the mouthpiece.

The support may comprise a hollow, longitudinally extending support portion and the mouthpiece may extend the hollow support portion. The hollow support portion can be grippable. The hollow support portion can feature reliefs and/or recesses shaped to ensure optimal gripping of the image taking device.

The image taking device is shaped so that, in the operating configuration, the hollow support portion is at least partly, preferably entirely, outside of the user's mouth.

The hollow support portion can be made of cardboard or, preferably, a thermoplastic material.

The hollow support portion can take the form of a longitudinally extending housing opening substantially outwards only through the mouthpiece opening, and optionally through a ventilation opening, for example longitudinally opposite the mouthpiece opening. The ventilation opening facilitates the evacuation of humid air exhaled by the user, in the operating configuration of the image taking device. The hollow support portion can be substantially cylindrical in shape.

The hollow support portion may comprise a bottom wall, for example solid, longitudinally opposite the mouthpiece opening. The image acquisition apparatus may be in contact with, or attached to, the back wall.

The hollow support portion can be sandwiched between the mouthpiece and the image acquisition apparatus.

Preferably, the hollow support portion defines the image taking chamber. Alternatively, the image acquisition apparatus, preferably according to the second embodiment described below, is remote from the image taking chamber.

The length of the hollow support portion, measured along the longitudinal axis, is less than 15 cm, preferably less than 10 cm.

At least the hollow support portion, or even the support, can be telescopic, i.e. its length, measured along its longitudinal axis, is variable. For example, it is made up of a plurality of parts that slide into one another.

In one variant, the support further comprises a grippable support portion which is mounted on the hollow support portion, so that the device can be handled by gripping the grippable support portion.

The grippable support portion can extend longitudinally and protrude transversely from the hollow support portion.

Preferably, the hollow support portion is arranged between the mouthpiece and the grippable support portion.

The hollow support portion can be attached, for example removably, to the grippable support portion. Preferably, the grippable support portion and the hollow support portion form a monolithic assembly.

The grippable support portion can define a cavity and include a cover closing the cavity. The device may comprise a printed circuit board and/or a battery controlling and/or electrically powering the image acquisition apparatus, which are preferably accommodated in the cavity. The cavity can also be made watertight, in particular by means of one or more seals.

Furthermore, the battery may be self-contained. It can be electrically charged by induction, or by a wired connection, such as USB. It can be powered directly by a wired electrical connection to an external power source.

The hollow support portion and/or the grippable support portion can be covered at least in part by a flexible material that facilitates gripping, for example a thermoplastic elastomer known as “TPE”, in particular a thermoplastic rubber known as “TPR”, such as styrene-butadiene-styrene, or a thermoplastic polyurethane known as “TPU”.

The mouthpiece is preferably shaped to move, and preferably curl, the user's upper and lower lips up and down respectively, in an operating configuration of the image taking device in which the mouthpiece is introduced into the mouth of the image taking device user, so as to maximize the dental surface visible to the image acquisition apparatus. “Up” and “down” refer to directions along a vertical axis when the user of the image taking device is standing upright. Preferably, the mouthpiece is shaped so that, when in at least one position of the operating configuration of the image taking device in which the mouthpiece is inserted into the user's mouth, the upper and lower lips are moved, preferably curled, upwards and downwards respectively so that at least the incisors, and preferably at least part of the canines and/or molars, of the upper and lower dental arches respectively are visible to the camera.

The mouthpiece can be integral with the support, in particular with the hollow support portion. In other words, the mouthpiece and the support, in particular the hollow support portion, can be obtained together in the same manufacturing operation. The hollow support portion and the mouthpiece can be produced by molding and/or additive manufacturing.

Alternatively, the mouthpiece can be mounted on the hollow support portion. It can be attached to the support, in particular to the hollow support portion. For example, it is snapped, screwed, or glued onto the support, particularly the hollow support portion. Alternatively, the mouthpiece can be removably mounted on the support, in particular on the hollow support portion, for example by magnetic coupling, by means of a hook-and-loop fastener, or by form-fit between the support, in particular the hollow support portion, and the mouthpiece. Advantageously, it is possible to mount different mouthpieces of different sizes on the support, in particular on the hollow support portion, and thus choose the mouthpiece best suited to the morphology of each user's mouth.

Preferably, the surface area covered by the mouthpiece opening is greater than 2 cm², preferably greater than 3 cm², more preferably greater than 5 cm², more preferably greater than 10 cm² , or even greater than 15 cm², for example around 19 cm². Preferably, it is less than 30 cm², more preferably less than 25 cm². This area is measured transversely to the longitudinal axis. The contour of the mouthpiece opening can be delimited by observing the image taking device along the longitudinal axis, from a position opposite the image acquisition apparatus along the longitudinal axis.

The image taking device can include a mirror attached to the support so that the image acquisition apparatus acquires a composite image comprising a direct image of the mouthpiece opening and an image of the mouthpiece opening reflected by the mirror. Thus, in the service configuration, the image acquisition apparatus sees a composite image comprising a direct image of the teeth observed directly through the mouthpiece opening and a reflected image of the teeth reflected by the mirror.

Alternatively, the image taking device may comprise a light source arranged to shine through the mouthpiece opening.

The light source may comprise at least one lamp, in particular a light-emitting diode lamp, and/or a flash unit.

The light source is preferably located in the image taking chamber.

The image taking device may comprise a control module to monitor the properties of the radiation emitted by the light source, in particular as a function of the light radiation received through the mouthpiece opening. For example, the control module can be configured so that more than 50%, more than 90% or even substantially 100% of the radiation received by the mouthpiece opening comes from the light source.

In particular, the image taking device may comprise several light sources and several cameras, each light source being arranged to illuminate the optical field of a corresponding camera. The light source(s) can be accommodated in the housing.

In order to determine the color and/or translucency of the user's teeth, the image taking device may comprise a colorimetric target and/or a translucency target, respectively, attached to the support, with the image acquisition apparatus viewing said target(s).

In addition, the image taking device may further comprise one or more additional cameras which, in at least one configuration of the image taking device, can be positioned in the user's oral cavity to acquire at least one intra-oral view of the lower dental arch and/or the upper dental arch, facilitating, for example, the taking of images of the lingual surfaces of the teeth, interdental spaces, caries control, or palate control. In particular, the image taking device can comprise a telescopic arm that extends between a folded configuration and at least one unfolded configuration, with the additional camera mounted on the telescopic arm. Preferably, in the folded configuration, the additional camera(s) are fully accommodated in the bracket. Preferably, in the operating configuration of the image taking device and in the unfolded position of the arm, the additional camera(s) are positioned in the user's oral cavity to acquire at least one intra-oral view of the lower dental arch and/or the upper dental arch.

Furthermore, in a first embodiment described below, the image acquisition apparatus is housed, at least in part, in the image taking chamber. Preferably, it also has a volume of less than 40 cm³. In addition to the features described above, according to this first embodiment, the kit can have any of the following features.

Preferably, the camera is less than 15 cm, preferably less than 7 cm, preferably less than 6 cm and most preferably more than 2.5 cm from the dental retractor.

The dental retractor defines an upper bearing surface and/or a lower bearing surface for contacting and displacing the user's lower lip and/or upper lip, respectively, in the device's service position in which the mouthpiece is inserted into the user's mouth. The distance between the mouthpiece and the image acquisition apparatus is the smallest distance between the lower bearing surface of the mouthpiece and the image acquisition apparatus, or between the upper bearing surface of the mouthpiece and the camera.

The image acquisition apparatus may comprise an additional screen. Preferably, the image acquisition apparatus has no additional display and/or telephone communication unit. Preferably, the image acquisition apparatus is not embedded in a cell phone. Preferably, the image acquisition apparatus is different from a cell phone.

In a second embodiment, the image acquisition apparatus comprises an additional screen. In addition to the features described above, according to this second embodiment, the kit can have any of the following features.

In particular, the image taking device can be as described in EP 3 391 810 A1 or WO 2022/008533A1.

Specifically, the image acquisition apparatus is a cell phone. The cell phone can digitally store the computer program described above.

Preferably, the image acquisition apparatus is removably mounted on the support. In particular, prior to mounting on the support, the acquisition apparatus can be configured to detect the support, for example from a distance of less than 10 cm, so as to trigger the execution of the computer program.

For example, the carrier includes a detection element, such as an RFID or NFC chip, which can be detected by the image acquisition apparatus.

The display device can be configured to deactivate the display on the additional screen. In the variant where the image acquisition apparatus is a cell phone, the display device can be configured to disable access to the cell phone network, to prevent the user from being interrupted by a phone call during image acquisition.

The kit according to the second embodiment may comprise any of the features of the kit according to the first embodiment, and vice versa.

The invention further relates to a method of taking extra-oral images using the photographic kit described herein, the method comprising
a) gripping the support and positioning the mouthpiece in a user's mouth,
b) acquiring at least one image of the user's lower dental arch and/or upper dental arch by the image acquisition apparatus, and
c) displaying the image on the display device which is visible to the user.

The positioning of the image acquisition apparatus is thus greatly facilitated, since the user can directly visualize the image acquired by the image acquisition apparatus. The operator, who may be the user, can thus move the support to a desired and/or optimal acquisition position while viewing the corresponding image(s) of the dental arches on the display device. Preferably, the method further comprises the user moving, for example, the image taking device relative to the user's mouth as a result of observing the image displayed on the screen.

In step a), the positioning of the mouthpiece allows the user's lower and upper lips to be moved apart to improve visibility of the lower and upper dental arches respectively.

The gripping and/or positioning of the support can be performed by an operator other than the user.

In step b), the user can be guided through the execution of the computer program as described above. The acquisition can be triggered automatically when the mouthpiece is inserted into the user's mouth. The method may comprise measuring the light intensity in the image taking chamber, and triggering image acquisition when the light intensity falls below a predetermined minimum light intensity.

Image acquisition can be triggered by the user or by an operator other than the user, for example in response to information supplied by the control module. The user can trigger acquisition by issuing a control command that is read by the image taking and/or display device. The control command is, for example, the actuation of a pushbutton attached to the support, or pressure on a touch screen of the display device.

A plurality of images can be acquired in step b). They can be acquired in series, i.e. the image acquisition apparatus acquires the images successively, and in particular at a fixed acquisition frequency, following a single acquisition trigger. The images can be displayed according to step c) in real time on the display device.

Preferably, the display device is separated from the image acquisition apparatus by a distance of less than 5 m, or even less than 1 m, and preferably more than 20 cm.

Preferably, the method comprises processing the image(s) acquired in one position of the image acquisition apparatus and generating information, preferably for the user. Preferably, the information is displayed by the display device.

The invention will be better understood from the following detailed description and the attached drawing, wherein:

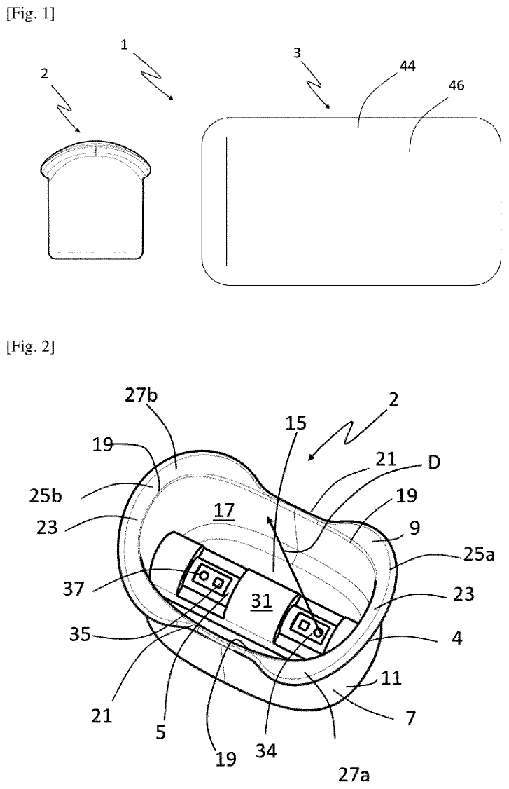

FIG. 1 shows an example of an image taking kit according to the invention,

FIG. 2 is a perspective view of an example of an image taking device,

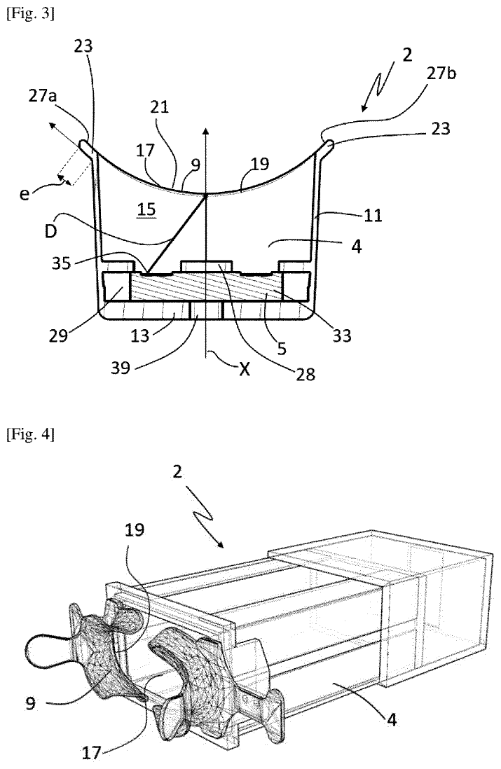

FIG. 3 is a cross-sectional view in a longitudinal plane of the image taking device shown in FIG. 2,

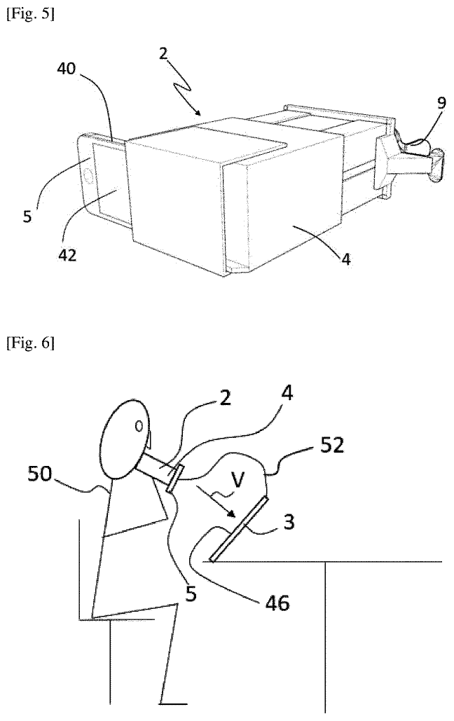

FIG. 4 and FIG. 5 are perspective views from different vantage points of another example of an image taking device,

FIG. 6 schematically illustrates an example implementation of the image acquisition method using the kit according to the invention.

### DETAILED DESCRIPTION

FIG. 1 shows an example of an image taking kit 1 according to the invention.

The kit 1 comprises an image taking device 2 and a display device 3.

The image taking device 2 comprises a support 4 and an image acquisition apparatus 5. The image acquisition apparatus 5 can have a volume of less than 40 cm³, resulting in a particularly compact image taking device.

The support 4 comprises a hollow support portion 7. The device further comprises a mouthpiece 9.

The hollow support portion 7 has a generally elongated tubular shape extending along a longitudinal axis X. It comprises a hollow tubular portion 11 and a bottom wall 13 which closes off one longitudinal end of the tubular portion. The tubular part 11 and the bottom wall 13 delimit a image taking chamber 15 wherein the image acquisition apparatus 5 is housed.

The bottom wall 13 can be solid or have ventilation openings running through it, for example in the form of cylindrical holes and/or grooves. In one embodiment, the hollow support portion 7 may be free of such a bottom wall.

At least part of the tubular portion 11, or even the entire tubular portion as illustrated, preferably extends parallel to the longitudinal axis X, to facilitate the holding and handling of the hollow support portion.

The tubular section 11 has at least one cross-section transverse to the longitudinal axis X, with a generally oblong shape. Such a shape of the hollow support portion facilitates the gripping of the support and also substantially corresponds to the shape of the contour of the access opening to a user's oral cavity, which is delimited by the lower and upper lips when the user spreads their lower and upper jaws apart.

The tubular part 11 opens out at another end, longitudinally opposite the bottom wall, into a mouthpiece opening 17.

The contour 19 of the mouthpiece opening is shaped like a saddle. In other words, when viewed from two perpendicular directions which are themselves orthogonal to the longitudinal direction, the contour 19 of the mouthpiece opening 17 has two opposing convex portions 21 connected by opposing concave portions 23.

The shape of the mouthpiece opening is preferably adapted to the general shape of the user's dental arches.

The mouthpiece 9 is attached to the hollow support portion 7. In the example shown, it is made of the same material as the hollow support portion. Other configurations can be envisaged, for example the mouthpiece being made of a different material than the hollow support portion and/or being mounted on the hollow support portion.

The mouthpiece 9 comprises two rigidly interconnected portions 25a-b, preferably symmetrical to each other with respect to a median plane, which respectively define an inner bearing surface 27a and an outer bearing surface 27b, for contacting and displacing the user's lower lip and/or upper lip respectively in the device's operating position in which the mouthpiece is introduced into the user's mouth.

The mouthpiece 9 is a flange that projects radially outwards from the tubular part 11, from the longitudinal end of the tubular part opposite the bottom wall 13.

The rim is ring-shaped and shaped like a saddle. The contour of the flange is homothetic with the contour of the mouthpiece opening.

The image acquisition apparatus 5 can be attached to the support 3. For example, as illustrated in FIGS. 2 and 3, the support comprises a receptacle 28 which defines a housing 29 of complementary shape to the image acquisition apparatus 5 and in which the image acquisition apparatus is received.

The receptacle comprises a receptacle wall 31, for example of hemi-cylindrical shape, which extends transversely between the opposite inner faces of the tubular part and longitudinally from the bottom wall of the hollow support portion.

The receptacle is integral with the hollow support portion. Alternatively, it can be attached to the hollow support portion.

The image acquisition apparatus comprises a housing 33 and two optical assemblies 34, each including a camera 35 and an LED lamp 37, which are housed entirely within the housing. This number of cameras and lamps is non-limiting.

The image acquisition apparatus 5 further comprises a connection socket 39 of the USB type, arranged in a recess in the thickness of the bottom wall 13. The socket allows the connection of a data transfer cable to transmit images taken through the mouthpiece opening 17 by the cameras 35 to the display device 3.

FIGS. 3 and 4 illustrate a variant of the image acquisition apparatus 3, as described in EP 3 391 810A1, wherein the mouthpiece 9 is removably mounted on the support 4, which can also be telescopic, and the image acquisition apparatus 5 is a cell phone 40 with a screen 42 removably mounted on the support and not directly visible to the user when the mouthpiece is inserted into the user's mouth.

In the example shown, the display device 3 is a digital tablet 44 comprising a screen 46, a memory for storing digital data, wherein a computer program for guiding the acquisition of images by means of the image acquisition apparatus 2 is loaded, and a processor for executing the computer program. However, other types of display device 3 are also possible. For example, the display device is a personal computer, a cell phone or a connected television, also known as a “smart TV”. In particular, the display device can be a cell phone and the acquisition device is another cell phone.

Images can be transferred between the image taking device and the display by means of a cable, one end of which is connected to the connection socket 39 on the image taking device shown in FIG. 2 or on the cell phone, and the other end to the connection socket on the digital tablet. Alternatively, the data, and in particular the images, can be transferred by wireless link, such as WIFI, between the image taking device and the display device.

FIG. 5 illustrates an example of how to implement the method according to the invention using the image taking kit according to the invention, for example as shown in FIGS. 1 to 4.

In order to acquire images of his dental arches, the user 50 takes hold of the support 4 and introduces the mouthpiece into their mouth, in order to curl their lips and uncover their arches so that they are visible to the image acquisition apparatus 5 through the mouthpiece opening.

The user can also order the execution of the computer program on the display device. For example, the execution of the program triggers image acquisition by the image acquisition apparatus 5 and real-time transfer of said images to the acquisition device via a connection cable 52. The user can thus directly view the image acquired by the image taking device 2, as indicated by the arrow V on the screen 46. As illustrated, the method can be implemented simply and conveniently for the user. Once the image has been acquired, the user can manipulate the support 4 to modify the position and orientation of the image acquisition apparatus relative to their face to acquire new images, which can then be viewed directly on the screen 46 and the quality of the acquisition assessed.

1) A image taking kit (1) comprising:
an extra-oral image taking device (2) comprising:
a support (4),
a mouthpiece (9) defining a mouthpiece opening (17) and carried by the support,
an image acquisition apparatus (5) fixed to the support and comprising a camera (35) for acquiring at least one image of a dental scene viewed through the mouthpiece opening (17), and
a display device (3) comprising a screen (46) for displaying an image representing the scene acquired by the image acquisition apparatus,
the image acquisition apparatus comprising an additional screen (42) and/or the support defining a photography chamber (15), the image acquisition apparatus being at least partially, and preferably entirely, housed within the photography chamber.

2) Kit according to claim 1, wherein the display device is selected from a mobile phone, a digital tablet, a desktop computer, in particular a portable one, and a television, in particular a connected one.

3) Kit according to any one of claims 1 and 2, comprising a communication module for transferring images acquired by the image taking device (2) to the display device (3).

4) Kit according to the preceding claim, wherein the communication module is a wireless communication module, in particular via radio frequency.

5) Kit according to claim 3, wherein the communication module comprises a data transmission cable (52), for example of the USB type, for connecting the image taking device (2) to the display device (3), and wherein the image taking device and the display device each comprise a connection port for plugging in the data transmission cable.

6) Kit according to any one of the preceding claims, comprising a control module for triggering the acquisition of images.

7) Kit according to the preceding claim, wherein the control module comprises a computer program comprising instructions for guiding the user to position the image taking device in at least one image acquisition position, and a processor for executing the computer program.

8) Kit according to the preceding claim, wherein the computer program comprises instructions for processing the acquired image or images in order to provide at least one piece of information to the user.

9) Kit according to any one of the preceding claims, wherein the image acquisition apparatus may comprise several cameras (35), in particular arranged along different viewing axes and each configured to acquire different views of the same dental scene.

10) Kit according to any one of the preceding claims, wherein the image acquisition apparatus has a volume of less than 40 cm³.

11) Kit according to any one of the preceding claims, wherein the area of the surface covered by the mouthpiece opening is greater than 2 cm².

12) Kit according to any one of the preceding claims, wherein the support comprises a hollow support portion (11) extending longitudinally, the mouthpiece (9) extending from the hollow support portion.

13) Kit according to the preceding claim, wherein the mouthpiece (9) is formed integrally with the hollow support portion or is mounted, in particular removably, on the hollow support portion (11).

14) Kit according to any one of the preceding claims, wherein the camera (35) is located less than 15 cm, preferably less than 7 cm, preferably less than 6 cm, and preferably more than 2.5 cm from the mouthpiece (9).

15) Kit according to any one of the preceding claims, wherein the image acquisition apparatus (5) is not embedded in a mobile phone and/or is different from a mobile phone.

16) Kit according to any one of claims 1 to 13, wherein the image acquisition apparatus comprises an additional screen (42), in particular wherein the image acquisition apparatus is a mobile phone (40).

17) An extra-oral image taking method using the image taking kit according to any one of the preceding claims, the method comprising:
a) gripping the support (4) and positioning the mouthpiece (9) in the mouth of a user (50),
b) acquiring at least one image of the lower dental arch and/or the upper dental arch of the user by the image acquisition apparatus (5), and
c) displaying the image on the display device (3) which is visible to the user (50).
